(19) United States
(12) Patent Application Publication
(10) Pub. No.: US 2019/0106453 A1
(43) Pub. Date: Apr. 11, 2019

(54) COMPLEXES OF NUCLEIC ACID MOLECULES AND METALS

(71) Applicants: Yissum Research Development Company of the Hebrew University of Jerusalem Ltd., Jerusalem (IL); Ramot at Tel-Aviv University Ltd., Tel Aviv (IL)

(72) Inventors: Danny PORATH, Jerusalem (IL); Dvir MAROM ROTEM, Jerusalem (IL); Alexander KOTLYAR, Tel Aviv (IL); Genady EIDELSHTEIN, Tel Aviv (IL)

(73) Assignees: Yissum Research Development Company of the Hebrew University of Jerusalem Ltd., Jerusalem (IL); Ramot at Tel-Aviv University Ltd., Tel Aviv (IL)

(21) Appl. No.: 16/078,392

(22) PCT Filed: Feb. 28, 2017
(86) PCT No.: PCT/IL2017/050253

§ 371 Date: Aug. 21, 2018

Related U.S. Application Data
(60) Provisional application No. 62/301,039, filed on Feb. 29, 2016.

(51) Int. Cl. C07H 21/04; C07H 23/00; H01B 1/12

(52) U.S. Cl. CPC C07H 21/04; H01B 1/121; C07H 23/00

(57) ABSTRACT

Provided is a conductive nucleic acid-metal complex including a polyG and PolyC consisting nucleic acids associated with a plurality of metal atoms, and methods for its preparation.

### TECHNOLOGICAL FIELD

The invention generally concerns conductive nucleic acid-metal complexes, methods of preparation and uses thereof.

### BACKGROUND OF THE INVENTION

Since the discovery of the chemical structure of DNA by Watson and Crick in 1953, significant knowledge has been accumulated about this unique molecule. The ability of DNA molecules to self-assemble into various, two- and three-dimensional nano-architectures [1-3] extended the field of DNA research well beyond biology. If DNA molecules would conduct electricity, a new generation of DNA-based circuits and electrical devices could be easily manufactured. However, the conductivity of the canonical double stranded (ds) DNA is very low, especially when the molecules are deposited on hard substrates [4-11].

Several studies have already shown that charge transport through the molecule can be considerably enhanced by positioning of noble metal atoms along the DNA template [12,13]. The metallization usually involves binding of metal ions to a DNA template and further reduction of the ions embedded inside the double helix by various reductants [14-16]. In most cases, the metallization process yields linear chains of NPs or metal clusters on a DNA template. The presence of non-metalized fragments between adjacent NPs in the chain, however, harms the ability of the DNA-metal complexes to conduct electricity.

U.S. Pat. No. 8,227,582 [17] discloses a process for the direct and selective metallization of nucleic acids via metal nanoparticles produced in-situ.

U.S. Pat. No. 5,948,897 [18] discloses a nucleic acid complex having double-stranded sections with a domain of guanine nucleotides.

US Patent Application No. 20040110163 [19] discloses nanoelectric conductors and conductive organic molecules capable of electron transport for use in biosensors and other types of electronics, including semi-conductors, transistors and switches. The redox active ions are disposed in the internal cores of guanine tetraplexes and coordinating such tetraplexes enables electron transfer and conductivity as organic wires.

US Patent Application No. 20070275394 [20] discloses a nucleic acid nanostructure including: a substrate; a nucleic acid quadruplex immobilized on the substrate; a metal ion present in a unit lattice of the nucleic acid quadruplex, and a method of manufacturing the same.

US Patent Application No. 20160287152 [21] discloses a nanoparticle conjugate which includes a nanoparticle, first oligonucleotides of one or more types bound to the nanoparticle and targeting conjugates of one or more types.

U.S. Pat. No. 7,419,818, [22] discloses an organic conductor comprising a DNA and an electric charge-donating material bonded to the DNA.

US Patent Application No. 20060257873 [23] discloses organic circuit elements and organic conductors, together with electron acceptors and donors that may be chemically modified to alter the conductivity of the circuit or organic conductor.

U.S. Pat. No. 7,160,869 [24] discloses organic circuit elements that include a plurality of members, each of which includes an oligonucleotide duplex.

US Patent Application No. 20060246482 [25] discloses linker molecules comprising one or more nucleic acid binding group and one or more nanoparticle binding group which are connected covalently by a spacer group.

US Patent Application No. 20040038229 [26] discloses various methods for enzymatically manipulating nanoparticle-bound nucleic acids.

U.S. Pat. No. 7,498,423 [27] discloses nucleic acid molecules in a stabilized solution such as single stranded DNA and RNA which are able to disperse high concentration of bundled carbon nanotubes into aqueous solution.

Shapir E, et al [28] reported on energy gap reduction in DNA by complexation with metal ions.

### BACKGROUND ART

[1] N. C. Seeman, Sci. Am. 2004, 290,
[2] N. C. Seeman, Annu. Rev. Biochem. 2010, 79, 65,
[3] P. W. K. Rothemund, Nature 2006, 440, 297,
[4] Braun, et al., Nature 1998, 391, 775,
[5] Porath, et al., Nature 2000, 403, 635,
[6] Watanabe, et al., Phys. Rev. Lett. 2001, 79, 2462,
[7] Cohen, et al., PNAS 2005, 102, 11589,
[8] de Pablo, et al., Phys. Rev. Lett. 2000, 85, 4992,
[9] Storm, et al., Appl. Phys. Lett. 2001, 79, 3881,
[10] Porath, et al., Topics in Current Chemistry. 2004, 237, 183,
[11] Livshits, et al., Nat. Nanotech. 2014, 9, 1040,
[12] Rakitin, P. et al., Phys. Rev. Lett. 2001, 86, 3670,
[13] J. Timper, et al., Angew. Chem. Int. Ed. Engl. 2012, 51, 7586,
[14] Molotsky, et al., J. Phys. Chem. C 2010, 114, 15951,
[15] Shemer, et al., J. Am. Chem. Soc. 2006, 128, 11006,
[16] Berti, et al., J. Am. Chem. Soc. 2005, 127, 11216,
[17] U.S. Pat. No. 8,227,582,
[18] U.S. Pat. No. 5,948,897,
[19] US Patent Application No. 20040110163,
[20] US Patent Application No. 20070275394,
[21] US Patent Application No. 20160287152,
[22] U.S. Pat. No. 7,419,818,
[23] US Patent Application No. 20060257873,
[24] U.S. Pat. No. 7,160,869,
[25] US Patent Application No. 20060246482,
[26] US Patent Application No. 20040038229,
[27] U.S. Pat. No. 7,498,423,
[28] Shapir E, et al, Adv Mater. 2011; 23(37), 4290-4.

### SUMMARY OF THE INVENTION

The inventors of the invention disclosed herein have developed a novel class of electrical (E) nucleic acid molecules, such as DNA, that are obtainable by contacting the nucleic acid, e.g., double stranded DNA with metal (e.g., silver) clusters.

The nucleic acid-metal complex of the invention is generally characterized by one or more of the following features:
It is a product of a metallization process that is selective to poly(dG)-poly(dC) strands and does not take place for poly(dA)-poly(dT) or essentially random sequences;
It is a product of a metallization process that is slow and therefore allows for better control over the metallization progress and resulting products, as compared with other metallization schemes known in the art;
The complex is about one third shorter than the double stranded nucleic acid molecule from which the complex is derived;
The complex has an AFM measurable apparent height that is about one third greater than that of the double stranded nucleic acid molecule from which the complex is derived. This is much lower than any reported metalized conductive dsDNA;
The complex is resistant to degradation by enzymes such as DNAses;
The complex is stable under ambient conditions;
The complex is more rigid and more resistant to mechanical deformation than the canonical ds nucleic acid molecule from which it is derived; and
The complex may be conductive, depending on the selection of metals.

These and other characteristics of complexes of the invention define a molecular nanowire that is useful for programmable electronic circuits and sensors. This is mainly based on the narrow but uniform width, the selective metallization and resistance to enzymes. Thus, the invention provides a nucleic acid metal complex that is not only producible under controlled conditions and thus permits controlled constructions of tailoring of structure and properties but also a complex that is highly usable and clearly superior to other similar conductive molecular entities.

The complex of the invention is not a nucleic acid tetraplex.

Thus, in a first aspect, there is provided a double-stranded nucleic acid-metal complex comprising:
a double stranded nucleic acid comprising at least one continuous region consisting of guanine (G) and cytosine (C) nucleotides, and
a plurality of metal atoms;

wherein said at least one continuous region is associated with the plurality of said metal atoms.

The nucleic acid part of the nucleic acid-metal complex is typically a double stranded molecule, not a tetraplex, comprising two complementary strands. The two strands are complementary anti-parallel strands that run in opposite directions alongside each other, allowing formation of hydrogen bonds such that paring between complementary nucleotide base pairs (e.g., between G and C) is possible. Accordingly, the two nucleic acid (e.g. DNA) strands are held together by inter-chain hydrogen bonds which pair the bases in one chain to the complementary bases in the other chain. The two strands may be both DNA, both RNA or a chimera of DNA and RNA strands.

One or both strands may optionally be modified to contain one or more special or modified bases (e.g., bases that have been modified after the nucleic acid chain has been formed or modified bases which have been used as building blocks in the construction of the nucleic acid chain) or other inserted molecular additions, e.g., functional groups permitting sensing, functional group connectors, linkers; and others as will be further described below. Non-limiting examples of modified nucleic acid bases that may be utilized include, for example, 2-aminopurine, 2,6-diaminopurine, 5-bromo dUridine, deoxyUridine, deoxyInosine, 5-hydroxybutynl-2′-deoxyuridine, 5-nitroindole, 5-methylcytosine (m5C), 8-aza-7-deazaguanosine, 5-methyl deoxycytidine, iso-dCytosine, iso-dGuanine, pseudouridine (Ψ), dihydrouridine, inosine, 7-methylguanosine, fluoro-substituted bases and others.

The “complex” comprising the nucleic acid and a plurality of metal atoms is a product of association between a region of the nucleic acid that consists of G and/or C nucleotides and a plurality of metal atoms. Without wishing to be bound by theory, the metal atoms are believed to be located between the nucleic bases. As the metal atoms are non-polar they tend to escape from the aqueous phase. However, as the nucleic acid core is apolar, it provides a favorable environment for the metal atoms. Hydrophobic forces are believed to be a factor stabilizing the complex.

The nucleic acid may comprise one or more continuous regions, i.e., one or more uninterrupted stretches of nucleotides, that is (are) essentially (consists) only G or C nucleoids or a combination of G and C nucleotides and which association with the plurality of metal atoms enables conductivity (depending on the particular metal used) along the continuous region. The nucleic acid may further comprise additional segments directly associated with said continuous region(s) that are not exclusively G or C, or that are not nucleic acids or which do not comprise any nucleotides. The additional segments may be at either or both termini of the nucleic acid strand(s) or interconnecting two or more continuous regions of G and C nucleotides.

In accordance with the present invention, the continuous regions may be interrupted by stretches of various lengths of various types of linker moieties, wherein any of linker moieties may comprise various combinations of A, T, G and C bases. In some cases, such linker moieties are free of A and/or T and/or G and/or C bases.

In some embodiments, the linker moiety may be short double stranded DNA fragments that contain restriction endonucleases cleavage sites.

In some embodiments, the linker moiety is not a nucleic acid.

In some embodiments, the linker moiety is an organic group that allows direct association of an atom or group along the continuous region with an atom or a group on a different continuous region or an additional segment. In some embodiments, such groups may be amines or N-containing groups.

The number and position of linker moieties along the nucleic acid part of a complex of the invention may vary and may depend on the particular purpose. The moieties may attach to either cohesive (sticky) or blunt ends of a continuous region(s) so as, e.g. to enable a point of connectivity to other moieties or functional or active groups or other continuous regions. For example, each of the nucleic acid strands may have a sticky end of a certain nucleic acid sequence which would enable association prior to or after the complex has been formed. The sticky end may be of a length and a sequence selected, inter alia, based on the association partner. Similarly, the sticky ends may be organic functional groups that permit association with a molecular entity of choice. For the purpose of a particular final utility, one or both strands of the double stranded nucleic acid, in a complex of the invention, may be appended with a linker moiety that would permit connectivity between the complex, once formed, and a surface region of a substrate or an entity selected from nanoparticles, nanotubes, nanorods, electric circuits, etc.

Each nucleic acid strand in a double stranded nucleic acid chain making up a complex of the invention comprises one or more continuous regions, such that continuous regions on each of the two anti-parallel nucleic acid strands match, affording paired strands. The ‘paired strands’ consist of preferably 100% base-pair matches (i.e. no mismatches at all), along at least one or the one or more continuous regions, whereby the identity of one strand determines the identity of the anti-parallel strand such that, in some embodiments, an equal number of G and C bases is present on the two strands of a continuous region, i.e., a G base is found opposite each C base in said a continuous region and vice versa.

In some embodiments, the paired strands consist 100%, 99%, 98%, 97%, 96% or 95% base-pair matches. In some embodiments, the paired strands consist of between 90 and 90% base-pair matches.

In some embodiments, the nucleic acid constitutes a double stranded nucleic acid molecule comprising a continuous region, in which (within said continuous region) one strand consists essentially of G and the other consists essentially of C nucleotide bases, to form paired strands (based on one strand being complementary to bases on the other anti-parallel strand). This embodiment is depicted below:

wherein a pair of anti-parallel continuous regions is depicted, one on each strand of the depicted double stranded nucleic acid, one region consisting G bases (Poly G) and the other consisting C bases (Poly C); each wavy line depicts an end group or a potential point of connectivity or a linker moiety, which may or may not be present. Each of the solid lines connecting between the two continuous regions depicts H-bonding present between the two strands.

In some embodiments, the nucleic acid constitutes a double stranded nucleic acid molecule comprising a continuous region, in which (within said continuous region) each of the two strands consists of a combination of G and C nucleotides, to form paired strands (based on one strand being complementary to bases on the other anti-parallel strand). This embodiment is depicted below:

wherein a pair of anti-parallel continuous regions is depicted, one on each strand of the depicted double stranded nucleic acid, one region consisting a combination of G bases and C bases and the other consisting a combination of C bases and G bases such that G in one continuous region is paired with C in the other; each wavy line depicts an end group or a potential point of connectivity or a linker moiety, which may or may not be present. Each of the solid lines connecting between the two continuous regions depicts H-bonding present between the two strands.

In other embodiments, the nucleic acid constitutes a double stranded nucleic acid molecule comprising a combination of two or more continuous regions, wherein along at least one of said two or more continuous regions, one strand consists essentially of G and the other consists essentially of C nucleotides, and along at least one other of said two or more continuous regions, each of the strands consists a combination of G and C nucleotides, to form paired strands (based on one strand being complementary to bases on the other anti-parallel strand). This embodiment is depicted below:

wherein two pairs of anti-parallel continuous regions are depicted, each as explained above. Each wavy line depicts an end group or a potential point of connectivity or a linker moiety, which may or may not be present. Each of the solid lines connecting between the two continuous regions depicts H-bonding present between the two strands.

In some embodiments, the double stranded nucleic acid consists:
one strand consisting G nucleotides; and
second strand consisting C nucleotides.

In another aspect, the invention provides a double stranded nucleic acid-metal complex comprising:
a double stranded nucleic acid comprising at least one continuous region, wherein along said continuous region one strand consists of G nucleotides and the other strand consists of C nucleotides, and
a plurality of metal atoms;
   wherein said at least one continuous region is associated with the plurality of said metal atoms.

In another aspect, the invention provides a double stranded nucleic acid-metal complex comprising
a double stranded nucleic acid comprising at least one continuous region, wherein along the continuous region, each strand consists of a combination of G and C nucleotides, in a sequence forming paired strands; and
a plurality of metal atoms;
   wherein said at least one continuous region is associated with a plurality of said metal atoms.

In another aspect, the invention provides a double stranded nucleic acid-metal complex comprising:
a double stranded nucleic acid comprising a combination of two or more continuous regions, wherein along at least one of said two or more continuous regions, one strand consists of G nucleotides and the other strand consists of C nucleotides, and along at least one other of said two or more continuous regions, each strand consists of a combination of G and C nucleotides, in a sequence complementary to each other; and
a plurality of metal atoms;
   wherein said at least one continuous region is associated with a plurality of said metal atoms.

The lengths of the full nucleic acid parts (in case the full nucleic acid is longer than the length of the continuous region and comprises it) of complexes of the invention may be from a few tens of nucleotides to a few thousands of nucleotides or even longer, depending on the intended use. In some embodiments, the number of nucleotides is between tens of nucleotides and several thousand nucleotides and the continuous region(s) may be positioned at any part of the nucleic acid molecule. Thus, according to the invention, the nucleic acid may comprise a number of continuous regions, e.g. each having a different length and different C/G composition, wherein the regions between each pair of continuous regions or between a continuous region and the end of the nucleic acid (5′ or 3′ end) may comprise A, T, C or G nucleotides and any combinations thereof.

In some embodiments, the length of the nucleic acid part of the complex is between about 10 and about 30,000 nucleotides. The length of the continuous region of the nucleic acid complex may be identical to the length of the nucleic acid part in case the complex does not contain regions outside or in addition to the so-called continuous regions, or may be of shorter lengths.

In some embodiments, the nucleic acid consists the continuous region.

In some embodiments, the nucleic acid comprises one or more continuous regions.

In some embodiments, the length of the continuous region is between about 10 and about 200 nucleotides. In other embodiments, the length of the continuous region is between about 50 and about 150 nucleotides. In some embodiments, the number of nucleotides is between 10 and 1,000, between 10 and 900, between 10 and 800, between 10 and 700, between 10 and 600, between 10 and 500, between 10 and 400, between 10 and 300, between 10 and 200, between 10 and 100, between 10 and 50, between 10 and 40, between 10 and 30, between 10 and 20, between 50 and 1,000, between 50 and 900, between 50 and 800, between 50 and 700, between 50 and 600, between 50 and 500, between 50 and 400, between 50 and 300, between 50 and 200, between 50 and 100, between 50 and 90, between 50 and 80, between 50 and 70, or between 50 and 60.

The metal atoms which are part of a complex of the invention, are neutral atoms (not ions) derived from metal particles or clusters or collections or aggregates of metal atoms. The “metal particles” are particles comprising or consisting a plurality of metal atoms (neutral atoms). In some cases, and depending on the intended uses, the metal need not be selected to provide conductivity. In some embodiments, the metal is selected amongst metals permitting conductivity along the nucleic acid complex of the invention. The metal particle may be in the form of an aggregate or a collection or a cluster comprising a plurality of metal atoms. The aggregate or collection or cluster of atoms may consist a single metal element from the periodic table or a mixture or combination of two or more such metal elements. The aggregate or collection of metal atoms may be in the form of a particle, e.g., a nanoparticle or a microparticle comprising a plurality of metal atoms.

In some embodiments, in a complex of the invention, the metal atoms, being neutral atoms, may be accompanied with an opportunistic amount of the corresponding metal ions. In some embodiments, the plurality of metal atoms that are part of a complex of the invention are neutral. In some embodiments, the plurality of metal atoms in a complex of the invention comprises an amount of the corresponding metal ions. Where metal ions are present, they are not the result of any addition or supplementation of metal ions but rather may be the product of a competing oxidative or reductive side reactions.

In some embodiments, the metal particle is a metal nanoparticle. In some embodiments, the metal nanoparticle consists a single type of a metal element.

In some embodiments, the metal particle is a metal nanoparticle, having at least one dimension in the nano-scale i.e., less than 1,000 nm. In some embodiments, the nanoparticle is a spherical nanoparticle, wherein the diameter of said spherical nanoparticle is less than 200 nm, less than 150 nm, less than 100 nm, less than 70 nm, less than 50 nm, less than 30 nm, less than 20 nm or is about 15 nm.

In some embodiments, the metal is a transition metal selected from Groups IIIB, IVB, VB, VIB, VIIB, VIIIB, IB and IIB of block d the Periodic Table. In some embodiments, the transition metal is selected from Sc, Ti, V, Cr, Mn, Fe, Ni, Cu, Zn, Y, Zr, Nb, Tc, Ru, Mo, Rh, W, Au, Pt, Pd, Ag, Mn, Co, Cd, Hf, Ta, Re, Os, Ir and Hg or any combination thereof.

In some embodiments, the metal is selected from Ag, Cu, Ni, Zn, Co, Cr and Fe.

In some embodiments, the metal is Ag.

In some embodiments, the metal nanoparticle consists Ag. In some embodiments, the metal nanoparticle comprises Ag.

The invention thus further provides, a double-stranded nucleic acid-metal complex comprising:
a DNA or RNA or a chimeric DNA-RNA comprising at least one continuous region consisting of guanine (G) and cytosine (C) nucleotides, and
a plurality of silver metal atoms;

wherein said at least one continuous region is associated with the plurality of said silver metal atoms.

In some embodiments, the metal particle, e.g., nanoparticle, used as a source for the metal atoms, is coated with or surface-associated with a plurality of molecular species, being in some embodiments ligands selected to shield the particles from aggregation or prevent the particles from precipitating from the reaction mixture. In some embodiments, the molecular species are selected from oligonucleotides, the oligonucleotides optionally comprising deoxyadenosine, deoxyguanosine, deoxycytidine, deoxyuridine and deoxythymidine. In some embodiments, the oligonucleotide is a 10-base oligonucleotide. In some embodiments, the 10-base oligonucleotide is selected from a 10C and 10T oligonucleotides. In some embodiments, the oligonucleotides is selected amongst oligonucleotides having between 5 and 20 bases.

In some embodiments, the metal particles consist of a plurality of atoms, e.g., silver atoms, in the form of a metal cluster or a multi-atom particle of between 1 atom and about 150 atoms. In some embodiments, the metal particles consist between 5 atom and about 100 atoms, or between 5 atom and about 90 atoms, between 5 atom and about 80 atoms, between 5 atom and about 70 atoms, between 5 atom and about 60 atoms, between 5 atom and about 50 atoms, between 5 atom and about 40 atoms, between 10 atom and about 100 atoms, between 10 atom and about 90 atoms, between 10 atom and about 80 atoms, between 10 atom and about 70 atoms, between 10 atom and about 60 atoms, between 10 atoms and about 50 atoms, between 50 atoms and about 150 atoms, between 60 atoms and about 120 atoms, between 80 atoms and about 120 atoms or between 70 atom and about 110 atoms.

In some embodiments, the metal particle has a size (diameter) of between about 0.34 nm and about 20 nm, or between 1 nm and 20 nm, or between 3 nm and 17 nm, or between 5 nm and 17 nm, or between 5 nm and 16 nm, or between 6 nm and 15 nm, or between 0.34 nm and 10 nm, or between 1 nm and 10 nm, or between 1 nm and 9 nm, or between 1 nm and 8 nm, or between 1 nm and 7 nm, or between 1 nm and 6 nm, or between 1 nm and 5 nm, or between 1 nm and 4 nm, or between 1 nm and 3 nm, or between 1 nm and 2 nm.

In some embodiments, the size is 0.5 nm, 0.75 nm, 1 nm, 1.25 nm or 1.5 nm.

The metal atoms (e.g., silver atoms) may be distributed along the length of the G and/or C consisting continuous region, in an even manner or may be concentrated in a specific sub-region within said continuous region.

In some embodiments, the nucleic acid-metal complex of the invention is about one third shorter than the ds nucleic acid from which the complex is derived, i.e., from a nucleic acid absent of said metal ions.

In some embodiments, the thickness or height or length of the short axis of the nucleic acid-metal complex is between about 0.3 nm and 8 nm, or between 0.3 nm and 6 nm, or between 0.5 nm and 5 nm, or between 1 nm and 5 nm, or between 1 nm and 3 nm, or between 1 nm and 2 nm.

In some embodiments, the thickness or height or length of the short axis of nucleic acid-metal complex is smaller than 15 nm, smaller than 10 nm, smaller than 7 nm, or smaller than 5 nm.

In some embodiments, the length (long axis) of the nucleic acid-metal complex is shorter than 1,500 nm, shorter than 1,200 nm, shorter than 1,000 nm, shorter than 800 nm, shorter than 400 nm, shorter than 350 nm, or shorter than 300 nm.

In some embodiments, the nucleic acid-metal complex of the invention has an AFM measurable apparent height that is about a third larger than that of the ds nucleic acid from which the complex is derived.

In some embodiments, the complex comprises or consists a single continuous region, as defined. In some embodiments, the complex comprises two or more continuous regions, as defined. In some embodiments, where a complex comprises two or more continuous regions, the regions may be identical or different. The regions may differ in length (i.e., the number of bases) or in the sequence of the bases (i.e., in case of a combination of G and C bases).

Where the complex of the invention comprises two or more continuous regions, as defined, or where a strand of multiple continuous regions is desired, each two such continuous regions may be associated to each other via a covalent bond or a linker moiety that is selected from a nucleic acid chain, an amino acid, or any organic or inorganic moiety. Where conductivity over the full length of the complex is required, the two or more continuous regions, each being independently conductive, may be associated with another via a conductive group or polymer.

The complexes of the invention exhibit conductivity and allow transport of electrical current along each continuous region(s). As such, the complexes of the invention may be regarded as conductive molecular wires comprising a nucleic acid-metal complex according to the invention. The wires of the invention may be utilized in the construction of a variety of circuits, electrical, optical and opto-electronic devices or parts of sensing elements. The wires may further be assembled into an assembly of wires and may be constructed such that conductivity along the assembly may be tuned or controlled. In some embodiments, the wire of the invention may be used as a conductive interconnect or a network of such conductive interconnects.

In some embodiments, the complex of the invention has a bandgap smaller than the bandgap of the canonical ds nucleic acid molecule from which it is derived. In some embodiments, the bandgap of the complex is at least 10%, at least 20%, at least 25%, at least 30, at least 35%, at least 40% or at least 50% smaller than the bandgap of the canonical ds nucleic acid molecule from which it is derived. In some embodiments the complex of the invention does not have a band gap.

The complex or wire may be used as a nanowire and may be a part of a sensor or a sensor array or a larger circuit or electronic construct or device. Due to the complementary, self-assembly nature of the nucleic acid molecule making up a complex of the invention, complex 2D and 3D conductive architectures of nanowires may be constructed. Such architectures may be achievable by combining two or more conductive strands of the invention, as detailed above, or by using a complex having exposed (i.e. lacking metal), single stranded ends, for connecting to immobilized single stranded ends and then covering only the ends by the metal. In some embodiments, the complex 2D and 3D conductive architectures are achievable by metalizing a nucleic acid molecule after construction of the 2D and 3D architecture. In further embodiments, the 2D and 3D architectures may be achievable by folding a long nucleic acid molecule into 2D and 3D architectures. In some embodiments, folding a long nucleic acid molecule into 2D and 3D architectures comprises utilizing at least one short nucleic acid molecule to direct the folding of the long nucleic acid scaffold molecule.

Alternatively, the bare double stranded nucleic acid, composed of G and C nucleotides, may be constructed to have sticky ends of single nucleic acid strands, thereby permitting their assembly into the desired structure. The contacting with the metal atoms may follow in situ to produce a complex according to the invention.

Wires of the invention may be utilized in the construction or operation of a circuit, an electronic element, an optical element or an optoelectronic element. In some embodiments, the element is used in a device selected an electronic circuit, a diode, a transistor, a photodiode, a transmitter, a laser, a gain device, an amplifier, a switch, a marker, a bio-marker, a display, a large area display, liquid-crystal displays (LCDs), a detector, a photodetector, a sensor, a light emitting diode, a lighting system and a solar cell.

Thus, the invention further provides a device having at least one region thereof associated with a complex according to the invention. The device may be selected from a nanowire, a sensor, a sensor array, a larger circuit and an electronic construct.

In some embodiments, the device is selected an electronic circuit, a diode, a transistor, a photodiode, a transmitter, a laser, a gain device, an amplifier, a switch, a marker, a bio-marker, a display, a large area display, liquid-crystal displays (LCDs), a detector, a photodetector, a sensor, a light emitting diode, a lighting system and a solar cell.

In some embodiments, the device is a nano-electronic device.

In some embodiments, the device is a DNA-based programmable circuit.

The complex of the invention may be prepared to meet a particular utility or may be prepared as a generic wire for a variety of uses. Typically, the complex or wire of the invention may be prepared by contacting a double stranded nucleic acid with a cluster of metal atoms under conditions permitting migration of the metal atoms from said cluster to the nucleic acid bases and association of the metal atoms with the nucleotides along the continuous region consisting of G and/or C nucleotides. The process of the invention makes no use of an oxidizing or a reducing agent and commences with the contacting of the reaction species. Thus, in another one of its aspects, the present invention provides, a method of forming a complex according to the invention, the method comprising:
contacting a double stranded nucleic acid with at least one cluster of atoms (metal particle) of at least one metal (e.g., in solution) under conditions enabling interaction between the metal atoms and the double stranded nucleic acid; to thereby provide a metal coated double stranded nucleic acid.

The invention further provides a method of forming a complex according to the invention, the method comprising:
contacting a double stranded nucleic acid with at least one cluster of atoms (metal particle) of at least one metal, under conditions permitting said at least one cluster to dissociate into a plurality of metal atoms; to thereby provide a metal coated double stranded nucleic acid.

The metal particle is as defined hereinabove.

As used herein, “conditions enabling interaction” generally include conditions that allow the metal atoms to dissociate from the metal cluster, migrate along the nucleic acid and interact therewith. Typically, such conditions involve continuous contacting of the nucleic acid and the metal cluster, under, e.g. ambient temperature, and in a medium having a selected pre-defined pH etc. In some embodiments, the conditions involve contacting, in solution, the nucleic acid and the metal particle, e.g., at room temperature, and for a period of time sufficient to bring about the formation of a complex of the invention.

In some embodiments, the contacting is continued for a period of time between 5 hours and 2 weeks. In some embodiments, the contacting step is carried out over a period of between 5 hours and 12 hours, between 5 hours and 24 hours, between 5 hours and 36 hours, between 5 hours and 48 hours, between 5 hours and 60 hours, between 5 hours and 72 hours, between 5 hours and 84 hours, between 5 hours and 96 hours, between 5 hours and 108 hours, between 5 hours and 120 hours, between 5 hours and 132 hours, between 5 hours and 144 hours, between 5 hours and 156 hours, between 5 hours and 168 hours, between 5 hours and 180 hours, between 5 hours and 192 hours or between 5 hours and 240 hours.

In some embodiments, the contacting is continued for a period of 1, 2, 3, 4, 5, 6, 7, 8, 9, or 10 days; 1, 2, 3, 4, 5, 6, 7, 8, 9, or 10 weeks.

In some embodiments, the method is free of any step involving the use of one or more reducing agent and/or oxidizing agent.

In some embodiments, the method is free of any step involving the addition of metal ions, wherein the metal ions are of the metal species making up a complex of the invention.

The plurality of metal atoms that are associated with the continuous region of the nucleic acid are all derived from a metal particle, e.g., nanoparticle, or metal cluster that acts as a reservoir of metal atoms and which dissociates to provide the plurality of atoms. The dissociation into the plurality of metal atoms is typically spontaneous and may be governed or controlled by, e.g., tuning the ionic strength of the medium in which the metallization is carried out, the presence and identity of the ligand species associated with the metal particles, by the period of time the metallization process is permitted to run, etc.

In some embodiments, the double stranded nucleic acids may be further contacted with a further cluster of the same or different metal atoms so as to form an additional layer of the same or different metal on the first formed complex. In some embodiments, a complex formed in accordance with the invention may have one or more layers of metal atoms. In some embodiments, a complex of the invention may be covered with a layer of an organic or an inorganic material to endow the complex with at least one additional property, to tune one or more of the complex properties, to decay or diminish at least one property associated with the complex or to provide a protective coating to the complex. In some embodiments, the additional organic or inorganic coating is selected to increase conductivity of the complex.

In some embodiments, a complex of the invention may be modified or serve as a template for binding or associating elements or materials such as nanoparticles or nanoclusters, organic polymers (such as for example charged and uncharged polysugars), conductive polymers (such as poly-pyrroles, poly-anilines, poly-thiophenes, poly-indoles and others), and others.

In some embodiments, the method further comprises a step of sintering the metal atoms associated with the double stranded nucleic acid under conditions selected not to damage the double stranded nucleic acid or otherwise risk the integrity of the complex. The sintering conditions utilized may comprise the application of heat or flocculating agents. The sintering step may be carried at room temperature or at any other suitable temperature, depending, inter alia, on the metal to be sintered, the nucleic acid utilized, the size of the complex and other parameters known to a person of skill.

As may be appreciated, the process of the invention as well as the complex of the invention differ from processes and complexes or products of the art in at least the fact that the formation of the complex does not involve exposing a nucleic acid to an ionic solution or to another source of ions, but rather to atoms of a metallic element.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-F provide AFM images and statistical length analyses of ˜300 molecules of 2,000 bp poly(dG)-poly(dC) before (FIGS. 1A and 1C) and after (FIGS. 1B and 1D) incubation with the silver nanoparticles. The length values (FIGS. 1C and 1D), corrected for tip convolution by subtracting the molecule's apparent width (a good approximation for the tip diameter) from the measured length, yield an average length of 600±30 nm and 400±20 nm for the DNA molecules before and after incubation with silver nanoparticles, respectively. The change in height was measured on a large number of cross-sections taken by AFM at various sites along 1500 bp long similarly prepared molecules that were co-deposited on the same substrate (E). Each cross-section is taken along a line scan, measuring the two types of molecules simultaneously. For the points in (FIG. 1E) the heights on each molecule were averaged. The molecules' average heights vs. their measured lengths were correlated. Clearly, two distinguishable populations of molecules are observed (FIG. 1F).

FIGS. 2A-C depicts morphology evolution of the E-DNA formation. Poly(dG)-poly(dC) DNA (FIG. 2A) was incubated with AgNPs for: 3 (FIG. 2B) and 40 (FIG. 2C) hours. The same color scale is used in all the images. One can note the difference in height of segments along the molecules, corresponding to the height color bar. The E-DNA formation progresses mostly from the molecules termini towards the center, but not in a uniform rate and with some variation from molecule to molecule. Occasionally, it starts from a certain point on the molecule and progresses along the molecule. After 16 hours many of the molecules seem smooth, more rigid and uniform.

FIGS. 3A-C provide images: TEM (FIG. 3A) and SEM (FIGS. 3B-C) images of E-DNA. Elongated DNA-based molecules are observed as well as some roughly spherical features which are attributed to silver aggregates.

FIGS. 4A-B is a scheme of E-DNA formation (FIG. 4A) and AFM imaging of an intermediate stage of E-DNA formation (FIG. 4B). FIG. 4A—step (1): The AgNPs bind to DNA and donate its atoms to the nucleic acid. As a result, silver atoms and few atoms clusters are positioned within or on the DNA molecules. Step (2): The NPs dissociate, leaving some of their atoms bound to the DNA. Step (3): A number of binding-dissociation cycles yield E-DNA. FIG. 4B—The DNA was incubated with AgNPs for 20 h and imaged by AFM. AgNPs bound to the DNA molecules are indicated by the arrows.

### DETAILED DESCRIPTION OF THE INVENTION

The complexes of the invention are referred to also as E-DNA (Electrical DNA) and are exemplified by the following non-limiting examples.

Incubation of poly(dG)-poly(dC) DNA, with silver nanoparticles (NPs) yields uniform linear DNA-based molecules, which are thicker and shorter than the parent dsDNA, as shown by atomic force microscopy (AFM). The resulting DNA-based molecules are visible in transmission and scanning electron microscopy (TEM and SEM), in contrast to the parent dsDNA, which is invisible by both techniques. The morphological changes induced in the DNA can be completely reversed by incubation with dithiothreitol that strongly binds to silver atoms and ions. The morphology of neither poly(dA)-poly(dT) nor of random sequenced plasmid DNA are affected by the presence of the NPs. It is suggested that adsorption of silver atoms by the GC-sequences, which is termed here “metallization”, takes place during the incubation of the poly(dG)-poly(dC) dsDNA with the NPs. The selectively metalized hybrid DNA-based molecules conduct current and may be used as nanowires in nanoelectronic devices and DNA-based programmable circuits.

Atomic-force microscopy (AFM) imaging analysis demonstrates that incubation of oligonucleotide-coated 15 nm (in diameter) silver NPs (AgNPs) with poly(dG)-poly(dC) DNA yields uniform DNA-based molecules which length is shorter by one-third and which height is larger by about one-third than that of the parent DNA. The resulting DNA-based molecules are more rigid and more resistant to mechanical deformation than the canonical dsDNA. They can be visualized by TEM and SEM unlike the parent molecules, probably because of the metal atoms adsorption. The presence of silver atoms in the molecules is further indicated by X-ray photoelectron spectroscopy (XPS).

The dsDNA morphology can be recovered by complexation of the metal using dithiothreitol (DTT). The morphology evolution of formation and decomposition of the molecules was followed by AFM snapshot imaging. Moreover, their circular dichroism (CD) spectrum was changing upon transition and recovered upon treatment with DTT. The molecules were not digested by DNAse I, unlike the parent and recovered molecules. The transition of the dsDNA to E-DNA is sequence (GC) specific. Neither poly(dA)-poly(dT), nor random sequenced plasmid (Puc19) DNA undergo the transition during incubation with silver nanoparticles.

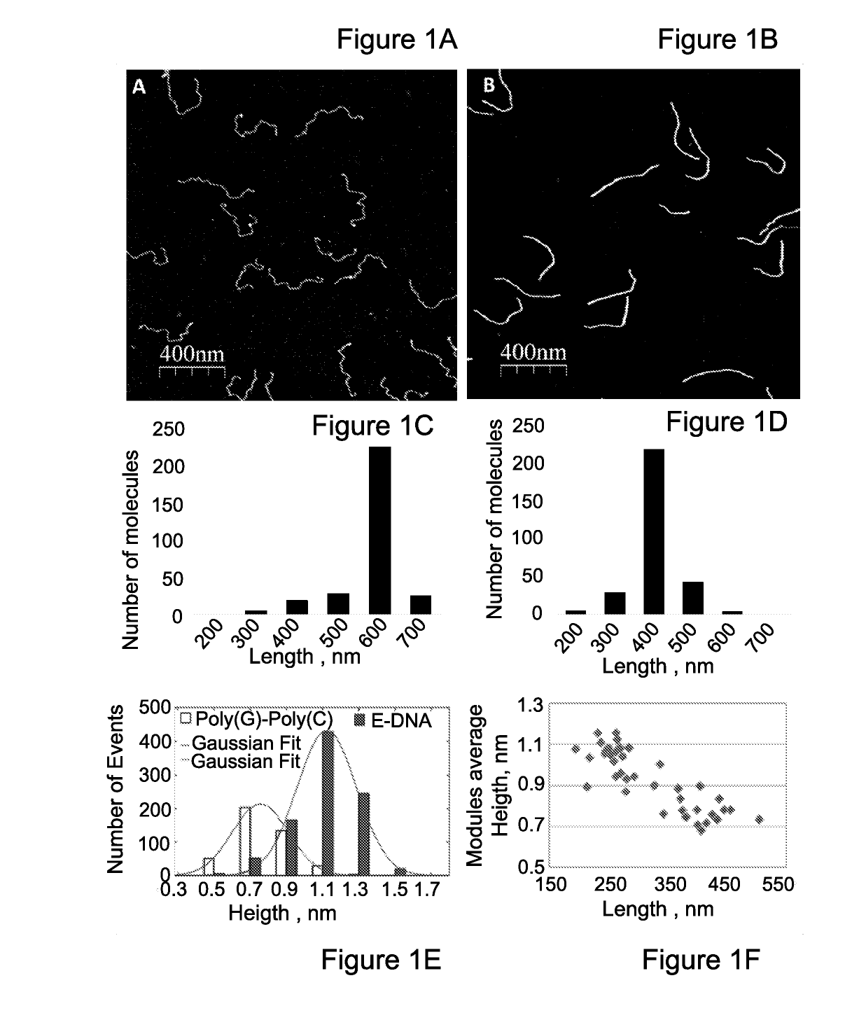

Incubation of poly(dG)-poly(dC) DNA with 15 nm (in diameter) spherical AgNPs coated with (dA)₁₀, an oligonucleotide composed of 10 deoxyadenosines, led to noticeable changes in morphology of the dsDNA, measured by AFM. It is clearly seen that DNA-based molecules obtained by the incubation are smooth, uniform, less wavy and more rigid as compared to the parent DNA (compare FIGS. 1A and 1B). The contour length of a 2,000 base pair long poly(dG)-poly(dC) DNA (FIG. 1A) was 600±30 nm (FIG. 1C). The contour length was reduced by approximately one-third during incubation of the DNA with AgNPs (compare the length histograms in FIGS. 1C and 1D). This shrinkage of the molecules was accompanied by an increase of their height from 0.7±0.1 nm to 1.1±0.1 nm (see FIG. 1E). A comprehensive height and height versus length analyses of a large number of cross-sections taken on a large number of similarly prepared 1500 bp long molecules, co-deposited on the same mica substrate, is shown in FIGS. 1E and 1F, respectively. Each cross-section is from the same scan line and taken simultaneously on the two types of molecules. The analysis reveals the height increase, as well as a clear height-length correlation within each one of the two types of molecules. Co-deposition enables a true comparison between both molecule populations, since their morphological features are not scan-dependent and attained under the same measuring conditions.

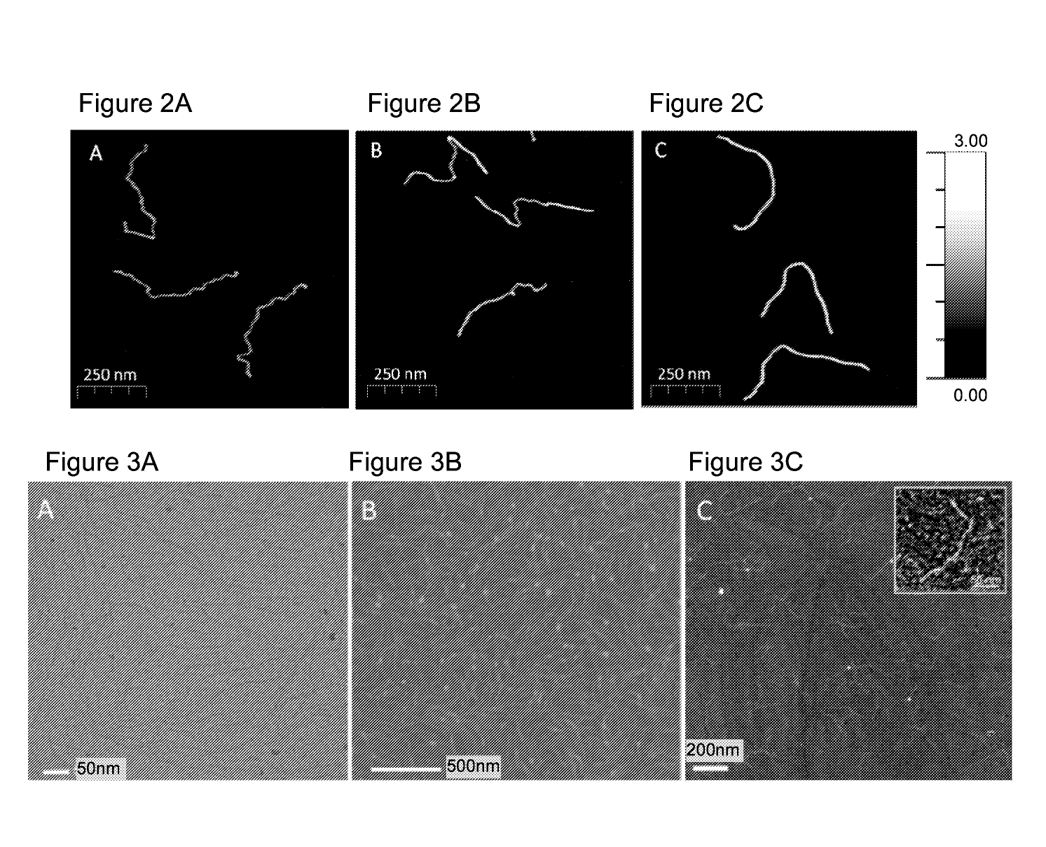

The process of E-DNA formation is gradual and takes about 2-3 days to completely convert poly(dG)-poly(dC) to E-DNA. FIG. 2 shows snapshots of the morphology evolution of the E-DNA formation, as depicted by AFM. Poly(dG)-poly(dC) is shown in FIG. 2A, and molecules that were incubated with AgNPs for 3 and 40 hours are shown in FIGS. 2B and 2C, respectively. The morphology change, observed in many molecules even after tens of minutes, progresses slowly on a time scale of hours (see FIG. 1). After 16 hours most of these molecules seem to adopt a nearly final morphological configuration. The transformation process is generally completed within 40 hours. A sharp difference in the height of different segments along the molecules, corresponding to dsDNA and E-DNA is clearly observed. In many cases, E-DNA formation starts at the DNA termini and progresses towards the center. Sometimes, however, it starts from a certain point on the molecule, possibly at the position of a structural defect, and progresses along the dsDNA.

The metallization process is selective to poly(dG)-poly(dC). The structure of neither poly(dA)-poly(dT), nor plasmid DNA (Puc19) is affected by incubation with the AgNPs. The process is therefore strictly selective and only GC-rich sequences undergo transition to E-DNA.

CD spectroscopy is a valuable method for studying the DNA conformation. Incubation with AgNPs led to a strong reduction of the signal amplitude in the 250-280 nm range of the spectrum. These data also support the suggestion that the poly(dG)-poly(dC) conformation has been changed during incubation. The new structure cannot, however, be interpreted from these spectra. E-DNA seems to be very stable: incubation with AgNPs at ambient temperature for two or even six months did not lead to any noticeable changes in the molecules morphology. The E-DNA structure seems to reach a thermodynamic equilibrium in the solution after a few days.

Incubation of E-DNA with DTT for various durations results in gradual but non-linear restoration of the dsDNA morphology. After 16 hours the dsDNA morphology is fully restored. It is known that DTT as well as other SH-containing compounds strongly bind silver atoms. The effect of DTT can thus be explained by assuming that the silver atoms that were bound to the E-DNA are scavenged by the dithiol. The molecule lacking Ag-atoms is then likely transformed back to the canonical dsDNA.

E-DNA is also resistant to DNAse in contrast to the parent DNA that is almost completely cleaved by the enzyme. The poly(dG)-poly(dC) that results from incubation of the E-DNA with DTT for 16 hours is completely cleaved by DNAse I, similar to the parent molecule as demonstrated by gel electrophoresis.

To verify the presence of silver atoms in E-DNA, elemental analysis was performed using XPS of molecules deposited on a cystamine modified flame annealed gold substrates. Clear peaks corresponding to silver are seen in the E-DNA sample in contrast to poly(dG)-poly(dC) and bare samples. A control sample, i.e. AgNPs incubated in the absence of DNA and centrifuged to remove the nanoparticles as described in the Experimental section, gave a 60% weaker signal than the E-DNA sample. These results suggest that the silver signal originates from E-DNA molecules and not from AgNPs that might be brought with the E-DNA solution.

The presence of metal atoms in the E-DNA was further supported by TEM and SEM analyses. FIG. 3 shows TEM (FIG. 3A) and SEM (FIGS. 3B and 3C) images of E-DNA. The TEM and SEM in FIGS. 3A and 3B were measured on the same grid. Elongated DNA-based molecules, which are 3-4 nm in diameter and about 200-400 nm long, were observed. No clear structures were observed in samples on which the same concentration of the parent DNA was deposited either by TEM or SEM. As a further control, E-DNA (approximately 1,200 bp long) was mixed with circular plasmid (puc 19) DNA in equal molar concentrations and imaged by both Cryo-TEM and SEM. Only linear molecules corresponding to the E-DNA length were observed by both techniques as shown in FIG. 3C. The poly(dG)-poly(dC) and the circular puc19 plasmids were not observed presumably due to their very low contrast. The visibility of the E-DNA is attributed to the presence of metal atoms which are likely to increase the contrast since they scatter electrons better than the light organic elements of the non-modified DNA.

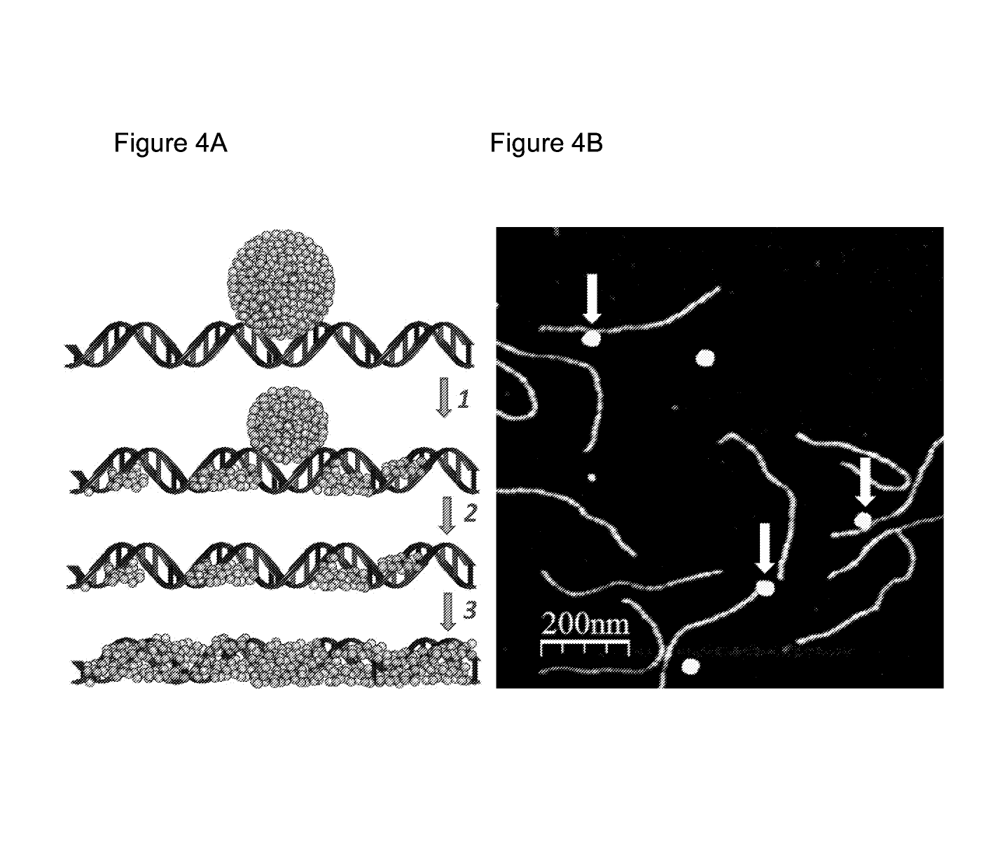

Taking together the results presented here, it is concluded that metallization of poly(dG)-poly(dC) molecules during incubation with the AgNPs takes place. Because in most molecules the transformation to E-DNA starts from the termini and sometimes from some central point, the termini and possibly sequence or other defects along the molecules are empirically more likely to bind AgNPs. The attachment probably triggers the metallization process speculated below, in which the silver is transferred to the molecule and the change of the molecular structure initiates. The directionality of the transition indicates that the gradual change along the molecule occurs base-pair by base-pair. Possibly metal atoms or few atoms clusters translocate to specific positions within or between the base-pairs, initiating the formation of a new equilibrium hybrid structure. The process progresses until the whole molecule is transformed to E-DNA. A tentative scheme, presented in FIG. 4A, illustrates the suggested process of E-DNA formation. The first step includes binding of the particle to the DNA molecule, either on its side or in the termini. AgNPs attached to the DNA are seen in many AFM images (see FIG. 4B), supporting the proposed step. It is unlikely that in the complex silver atoms are directly transferred from the NP to the DNA. The metallization process seems to involve oxidation of silver atoms on the surface of the NP by one of the bases, followed by their binding to the DNA molecule. Guanine, having the lowest ionization potential among the four nucleic bases is the most probable candidate for oxidation of silver atoms in the NP. In addition, the affinity of silver ions to G- and C-bases is higher than to A- and T-bases leading to specific binding of the metal ions to GC-rich DNA. Higher affinity of G and C bases to silver ions as compared to A and T ones together with the highest oxidation potential of guanines among the nucleic bases may account for the sequence-specific metallization of the DNA demonstrated here. After oxidation and binding to DNA a silver atom can get its electron back from the reduced guanine radical. A number of successive cycles of Ag atoms oxidation and transferring from the NP to the DNA results in positioning of the atoms in specific positions along the DNA or in the formation of few atoms silver clusters next to the site of the particle binding on the Poly(dG)-Poly(dC) molecule.

The metal atoms positioned along the DNA molecules improve the charge transport properties and make E-DNA an attractive candidate for nanoelectronics.

### Experimental Details

Unless otherwise stated, reagents were obtained from Sigma-Aldrich (USA) and were used without further purification. Klenow fragment exonuclease minus of DNA polymerase I from E. coli lacking the 3′-, 5′-exonuclease activity (Klenow exo⁻) was purchased from Epicenter Biotechnologies (USA) and puc 19 was from Thermo Fisher Scientific (USA).

### Oligonucleotide Purification and DNA Synthesis

All the DNA samples, A₁₀, C₁₂ and G₁₂ comprising 10 adenines, 12 cytosines and 12 guanines, correspondingly, were purchased from Alpha DNA (Montreal, Canada). Each oligonucleotide (˜1 mg) was dissolved in ˜200 μL of double distillate water (DDW) and subsequently passed through a pre-packed Sephadex G-25 DNA-Grade column (Amersham, Biosciences) equilibrated with 2 mM Tris-acetate, pH 7.5. The oligonucleotide eluted in the void volume, was collected in 0.4-0.5 mL and purified by ion-exchange HPLC to homogeneity.

### Enzymatic Synthesis of DNA

A standard reaction mixture contained: 60 mM K-Pi (pH 6.5), 5 mM MgCl₂, 5 mM DTT, 1.5 mM dGTP, 1.5 mM dCTP, 0.2 μM Klenow exo⁻, and HPLC purified template-primers, (dG)₁₂-(dC)₁₂. The enzymatic reaction was conducted for 1-2 h at 37° C. and was halted by the addition of EDTA to a final concentration of 10 mM.

### HPLC Purifications

The separation of synthesized DNA molecules from nucleotides and other reaction components was on a TSK-gel G-DNA-PW HPLC column (7.8×300 mm) from TosoHaas (Japan) by isocratic elution with 20 mM Tris-acetate (pH 7.5) for 30 min at a flow rate of 0.5 mL/min. The purification was conducted on a Finnigan Surveyor LC (Thermo Electron Corporation, USA) HPLC system with a photodiode array detector. Peaks were identified from their retention times obtained from the absorbance at 260 nm for DNA. Eluted products were concentrated by Amicon Ultra-30K-50K MWCO filter devices (Millipore, USA). The length of the synthesized molecules was determined by 1.5% Agarose gel electrophoresis.

### Synthesis of AgNPs

Spherical silver NPs with a diameter of 15±2 nm were prepared by AgNO₃ reduction in the presence of citric acid and borohydride (NaBH₄) as follows: 180 mL of DDW/filtered water were added into a 0.5 L glass beaker standing in an ice-water bath. 0.45 mL of 0.1M AgNO₃, 0.90 mL of 50 mM sodium citrate and 0.75 mL of 0.6 M NaBH₄ were consequently added into the beaker under vigorous stirring. The yellow solution was stored at 4° C. for 12-16 h. 0.72 mL of 2.5 M LiCl were then added under constant stirring at ambient temperature. The solution was transferred into 15 mL capacity DuPont Pyrex tubes and centrifuged at 14,000 rpm for 1.5 h at 20° C. in a Sorval SS-34 rotor. A fluffy pellet was collected.

Coating of AgNPs with (dA)₁₀, an oligonucleotide composed of 10 deoxyadenosines, was conducted at ambient temperature as follows: 20 μM (dA)₁₀ was added to 4 mL of AgNPs (OD˜90 at 400 nm). The mechanism of the oligonucleotide binding includes interaction of the nucleic bases with the surface of nanoparticles. The oligonucleotide-coated nanoparticles are relatively stable in aqueous solutions and do not aggregate even at relatively high salt concentrations (up to 150 mM) in contrast to citrate-protected nanoparticles. (dC)₁₀ and (dG)₁₀ oligonucleotides produce similar effect on the nanoparticles stability. In contrast to the above oligonucleotides, incubation of citrate-protected NPs with (dT)₁₀ does not yield stable nanoparticles. The coating procedure includes stepwise increase of the NaCl concentration during the incubation with the oligonucleotides. First the particles were treated with 20 μM (dA)₁₀ for 1 h in 25 mM NaCl at room temperature (RT); then the salt concentration was increased to 50 mM and the incubation was continued for additional 16 hours. Finally NaCl concentration was adjusted to 100 mM and the solution was incubated for 2 more hours and subsequently loaded onto a Sepharose 6B-CL column (1.6×35 cm). Elution was done with 10 mM Na-Pi (pH 7.4). The yellow eluate was collected into Eppendorf tubes and centrifuged at 13,000 rpm for 40 mM at RT on bench-top centrifuge 5424 (Eppendorf, Germany). The fluffy pellet was suspended by pipetting and stored in dark at ambient temperature. The resulting AgNPs were screened for their size and uniformity by TEM, revealing an average diameter of 15±2 nm. The visible spectra showed a characteristic absorption peak at 400 nm. The concentration of the NPs was calculated using an extinction coefficient (ε) of 2×10⁹ Mol⁻¹ cm⁻¹ at 400 nm.

### Preparation of E-DNA

200 μM (expressed in base pairs), poly(dG)-poly(dC) ranging in length from 1000 to 2000 bp was incubated with (dA)₁₀-coated AgNPs (OD at 400 nm˜30) in 5 mM Na-Pi (pH=7.5) containing 100 mM NaCl for 2-4 days at RT. AgNPs coated with (dC)₁₀ and (dG)₁₀ can be used for preparation of the E-form as well. The NPs were separated from the DNA by centrifugation for 5 min at 50,000 rpm on an ultracentrifuge (Beckman Coulter Optima TLX, Rotor—TLA-120.1) at 18° C. The supernatant was collected and stored at RT.

### Atomic Force Microscopy

AFM imaging was performed on molecules adsorbed on muscovite mica surfaces. 100 μL aliquots of 0.2 μM (in base pairs) DNA solution in 1 mM MgCl₂, were deposited on freshly cleaved 0.5×0.5 cm mica plates for 5 min. The surface was then washed with ultra-pure distilled water and dried by a nitrogen flow. AFM imaging was performed with two AFM systems: a Solver PRO AFM system (NT-MDT, Russia), in a semi-contact (tapping) mode, using 130 μm Si-gold-coated cantilevers (NT-MDT, Russia) with a resonance frequency of 100-120 kHz, and an Aist-NT SmartSPM AFM system, in AC (tapping) mode, using 240 μm Medium-Soft Silicon cantilevers (Olympus) with a resonance frequency of 60-80 kHz. The images were “flattened” (each line of the image was fitted to a second-order polynomial, and the polynomial was then subtracted from the image line) by the Nova image processing software (NT-MDT, Russia). The images were analyzed and visualized using a Nanotec Electronica S.L (Madrid) WSxM imaging software.

### CD Spectroscopy

The spectra were recorded with an Aviv Model 202 series (Aviv Instrument Inc., USA) CD Spectrometer. Each spectrum was recorded from 220 to 350 nm and was an average of 4 measurements. Recording specifications were: wavelength step 1 nm, settling time 0.333 sec, average time 1.0 sec, bandwidth 1.0 nm, path length 1 cm.

### X-Ray Photoelectron Spectroscopy (XPS)

Prior to the DNA deposition flame-annealed gold substrates were treated with cystamine as follows: the substrate was immersed into 0.5 mL of 10 mM cystamine solution and left for 24 hours. The substrate was then rinsed with distilled water and dried with a flow of nitrogen gas. This treatment introduces positive charges (amino groups) to the surface and promotes binding of a negatively charged DNA. A drop of a sample solution containing DNA was poured on the surface and incubated for 40 min. The surface was then washed with ultra-pure distilled water and dried by a nitrogen flow. The X-ray Photoelectron Spectroscopy (XPS) measurements were performed on a Kratos Axis Ultra X-ray photoelectron spectrometer (Karatos Analytical Ltd., Manchester, UK). High resolution XPS spectra were acquired with monochromatic Al Kα X-ray radiation source (1,486.6 eV) with 90° takeoff angle (normal to the analyzer). The pressure in the chamber was 1.8·10⁻⁹ Torr. The high-resolution XPS spectra were collected for Ag 3 d, N 1 s and C 1 s levels with pass energy 20 eV and step 0.1 eV. The samples were prepared on gold substrates, so for high resolution XPS analyses the gold peaks were not measured. Data analyses were performed using Casa XPS (Casa Software Ltd.) and Vision data processing program (Kratos Analytical Ltd.).

### TEM Measurements

Imaging was performed with a FEI Tecnai 12 G² Spirit TWIN TEM operated at an acceleration voltage of 120 kV, and images were recorded on a FEI Eagle 4K×4K CCD camera in low dose mode and with a 3-5 μm defocus.

For EM, 3 μL of sample was applied to a glow discharged ultrathin carbon on carbon lacey support film on 400 mesh copper grid (Ted Pella, Ltd). The excess liquid was blotted with a filter paper, and the grid was allowed to dry in air.

For Cryo-TEM, a drop (3 μL) of the solution was applied to a glow discharged TEM grid (300-mesh Cu grid) coated with a holey carbon film (Lacey substrate, Ted Pella, Ltd.). The excess liquid was blotted, and the specimen was vitrified by a rapid plunging into liquid ethane pre-cooled with liquid nitrogen using Vitrobot Mark IV (FEI).

The vitrified samples were examined at −177° C. using a FEI Tecnai 12 G² Spirit TWIN TEM equipped with a Gatan 626 cold stage, and the images were recorded (4K×4K FEI Eagle CCD camera) at 120 kV in low-dose mode.

### SEM Measurements

Scanning electron microscope (SEM) images of the same TEM grids to which the samples were applied and that were first observed by TEM were acquired using a FEI Magellan 400 L XHR SEM (without any further treatment).

A drop of water solution of GM was placed on a freshly cleaved HOPG and was subsequently removed from the surface with a flow of nitrogen in 5 min. 10 μL of the E-DNA solution was applied on a GM-treated HOPG surface. A drop of fresh DDW (100 μL) was gently placed above the drop of the sample solution and the liquid was removed from the surface with a flow of nitrogen. This sample was visualized by Zeiss Merlin with GEMINI 11 Electron Optics SEM.

1.-60. (canceled)

61. A double-stranded nucleic acid-metal complex, comprising:
a double stranded nucleic acid comprising at least one continuous region consisting guanine (G) and cytosine (C) nucleotides, and
a plurality of metal atoms;
wherein said at least one continuous region is associated with the plurality of said metal atoms.

62. The complex of claim 61, wherein within said continuous region one strand of the double strand nucleic acid consists essentially of G and the other strand consists essentially of C nucleotide bases, or within said continuous region each of the two strands of the double stranded nucleic acid consists a combination of G and C nucleotides.

63. The complex of claim 61, wherein the nucleic acid part of the complex comprises a combination of two or more continuous regions, wherein along at least one of said two or more continuous regions, one strand consists essentially of G and the other consists essentially of C nucleotides, and along at least one other of said two or more continuous regions, each of the strands consists a combination of G and C nucleotides.

64. The complex of claim 61, wherein the double stranded nucleic acid strand is DNA, RNA or a chimera of DNA and RNA.

65. The complex of claim 61, wherein the metal atom is a transition metal selected from Groups IIIB, IVB, VB, VIB, VIIB, VIIIB, IB and IIB of block d of the Periodic Table.

66. The complex of claim 65, wherein the metal is selected from Ag, Cu, Ni, Zn, Co, Cr and Fe.

67. The complex of claim 66, wherein the metal is Ag.

68. A double-stranded nucleic acid-metal complex, comprising:
a DNA or RNA or a chimeric DNA-RNA comprising at least one continuous region consisting of guanine (G) and cytosine (C) nucleotides, and
a plurality of silver metal atoms;
wherein said at least one continuous region is associated with the plurality of said silver metal atoms.

69. The complex according to claim 61, wherein the complex is about one third shorter than the double stranded nucleic acid from which the complex is derived.

70. The complex according to claim 61, wherein the complex has an AFM measurable apparent height that is about a third larger than that of the double stranded nucleic acid from which the complex is derived.

71. The complex according to claim 61, being conductive.

72. A nanowire comprising a complex of claim 61.

73. A method of forming a double-stranded nucleic acid-metal complex, the method comprising contacting a double stranded nucleic acid with at least one metal particle of at least one metal, under conditions permitting said at least one metal particle to dissociate into a plurality of metal atoms; to thereby provide a metal-coated double stranded nucleic acid.

74. The method of claim 73, wherein said at least one metal particle is in a form of an aggregate or a collection or a cluster comprising a plurality of metal atoms.

75. The method of claim 74, wherein said aggregate or collection or cluster of atoms consists a single metal element.

76. The method of claim 73, further comprising a step of sintering the metal atoms.

77. The method of claim 73, wherein the conditions permitting dissociation into a plurality of metal atoms comprise contacting the double stranded nucleic acid with the at least one cluster of atoms of at least one metal in solution, or the conditions permitting dissociation into a plurality of metal atoms comprise contacting the double stranded nucleic acid with the at least one cluster of atoms of at least one metal at room temperature, or the conditions permitting dissociation into a plurality of metal atoms comprise contacting the double stranded nucleic acid with the at least one cluster of atoms of at least one metal in solution, at room temperature.

78. The method of claim 73, wherein the double stranded nucleic acid is DNA, RNA or a chimeric DNA-RNA.

79. The method of claim 73, wherein the metal is silver.

80. A device wherein at least one region thereof is associated with a complex of claim 61.
